(19) United States
(12) Patent Application Publication
(10) Pub. No.: US 2013/0041424 A1
(43) Pub. Date: Feb. 14, 2013

(54) DUODENAL STIMULATION TO INDUCE SATIETY

(71) Applicant: Advanced Neuromodulation Systems, Inc., Plano, TX (US)

(72) Inventor: Hans Neisz, Coon Rapids, MN (US)

(73) Assignee: Advanced Neuromodulation Systems, Inc., Plano, TX (US)

(21) Appl. No.: 13/644,442

(22) Filed: Oct. 4, 2012

Related U.S. Application Data
(62) Division of application No. 12/712,106, filed on Feb. 24, 2010.
(60) Provisional application No. 61/154,989, filed on Feb. 24, 2009.

(51) Int. Cl. A61N 1/36

(52) U.S. Cl. 607/40

(57) ABSTRACT

Methods and devices for creating and/or adding to sensations of satiety to reduce food intake. Methods include electrically stimulating the duodenum which may induce false nerve signals in the duodenal region which are normally indicative of duodenal distension (fullness) and/or the presence of food in the duodenum. The artificially generated signals may be applied in a pattern which mimics at least in part a naturally occurring pattern of duodenal signals generated responsive to eating a meal. Some artificial patterns may be exaggerated relative to the natural patterns, by occurring earlier after ingestion, and/or lasting longer after ingestion, having an exaggerated (higher) frequency response or a faster rate of frequency increase after ingestion. The applied signals may generate nerve signals going to the brain which induce a feeling of satiety.

### CROSS REFERENCE TO RELATED APPLICATION

This application is a divisional of U.S. application Ser. No. 12/712,106, filed Feb. 24, 2010, pending, which claims priority from U.S. Provisional Application 61/154,989, filed Feb. 24, 2009, the disclosures of which are fully incorporated herein by reference for all purposes.

### TECHNICAL FIELD

The present invention is related generally to implantable medical devices and methods for treating obesity. More specifically, the invention relates to implantable electrical stimulation devices for stimulating nerves associated with the duodenum to generate neurological signals indicating duodenal distension to the autonomic nervous system.

### BACKGROUND

Obesity is an epidemic in the U.S. with a prevalence of about 20 percent. Annual U.S. healthcare costs associated with obesity are estimated to exceed $200 billion dollars. Obesity is defined as a body mass index (BMI) that exceeds 30 kg/m². Normal BMI is 18.5-25 kg/m², and overweight persons have BMIs of 25-30. Obesity is classified into three groups: moderate (Class I), severe (Class II), and very severe (Class III). Patients with BMIs that exceed 30 are at risk for significant comorbidities such as diabetes, heart and kidney disease, dyslipidemia, hypertension, sleep apnea, and orthopedic problems.

Obesity results from an imbalance between food intake and energy expenditure such that there is a net increase in fat reserves. Excessive food intake, reduced energy expenditure, or both may cause this imbalance. Appetite and satiety, which control food intake, are partly controlled in the brain by the hypothalamus. Energy expenditure is also partly controlled by the hypothalamus. The hypothalamus regulates the autonomic nervous system of which there are two branches, the sympathetic and the parasympathetic. The sympathetic nervous system generally prepares the body for action by increasing heart rate, blood pressure, and metabolism. The parasympathetic system prepares the body for rest by lowering heart rate, lowering blood pressure, and stimulating digestion.

Experimental and observational evidence suggests that there is a reciprocal relationship between food intake and sympathetic nervous system activity. Increased sympathetic activity reduces food intake and reduced sympathetic activity increases food intake. Certain peptides (e.g. neuropeptide Y, galanin) are known to increase food intake while decreasing sympathetic activity. Others such as cholecystokinin, leptin, enterostatin, reduce food intake and increase sympathetic activity. In addition, drugs such as nicotine, ephedrine, caffeine, subitramine, and dexfenfluramine increase sympathetic activity and reduce food intake.

Ghrelin is another peptide that is secreted by the stomach that is associated with hunger. Peak plasma levels occur just prior to mealtime, and ghrelin levels are increased after weight loss. Sympathetic activity can suppress ghrelin secretion. PYY is a hormone released from the intestine that plays a role in satiety. PYY levels increase after meal ingestion. Sympathetic activity can increase PYY plasma levels.

Appetite is stimulated by various psychosocial factors, but is also stimulated by low blood glucose levels. Cells in the hypothalamus that are sensitive to glucose levels are thought to playa role in hunger stimulation. Sympathetic activity increases plasma glucose levels. Satiety is promoted by distention of the stomach and delayed gastric emptying. Sympathetic activity reduces gastric and duodenal motility, causes gastric distention, and can increase pyloric sphincter, which can result in distention and delayed gastric emptying.

The sympathetic nervous system plays a role in energy expenditure and obesity. Genetically inherited obesity in rodents is characterized by decreased sympathetic activity to adipose tissue and other peripheral organs. Catecholamines and cortisol, which are released by the sympathetic nervous system, cause a dose-dependent increase in resting energy expenditure. In humans, there is a reported negative correlation between body fat and plasma catecholamine levels. Overfeeding or underfeeding lean human subjects has a significant effect on energy expenditure and sympathetic nervous system activation. For example, weight loss in obese subjects is associated with a compensatory decrease in energy expenditure, which promotes the regain of previously lost weight. Drugs that activate the sympathetic nervous system, such as ephedrine, caffeine and nicotine, are known to increase energy expenditure. Smokers are known to have lower body fat stores and increased energy expenditure.

The sympathetic nervous system also plays an important role in regulating energy substrates for increased expenditure, such as fat and carbohydrate. Glycogen and fat metabolism are increased by sympathetic activation and are needed to support increased energy expenditure.

Animal research involving acute electrical activation of the splanchnic nerves under general anesthesia causes a variety of physiologic changes. Electrical activation of a single splanchnic nerve in dogs and cows causes a frequency dependent increase in catecholamine, dopamine, and cortisol secretion. Plasma levels can be achieved that cause increased energy expenditure. In adrenalectomized anesthetized pigs, cows, and dogs, acute single splanchnic nerve activation causes increased blood glucose and reduction in glycogen liver stores. In dogs, single splanchnic nerve electrical activation causes increased pyloric sphincter tone and decrease duodenal motility. Sympathetic and splanchnic nerve activation can cause suppression of insulin and leptin hormone secretion.

First line therapy for obesity is behavior modification involving reduced food intake and increased exercise. However, these measures often fail and behavioral treatment is supplemented with pharmacologic treatment using the pharmacologic agents noted above to reduce appetite and increase energy expenditure. Other pharmacologic agents that can cause these affects include dopamine and dopamine analogs, acetylcholine and cholinesterase inhibitors. Pharmacologic therapy is typically delivered orally and results in systemic side effects such as tachycardia, sweating, and hypertension. In addition, tolerance can develop such that the response to the drug reduces even at higher doses.

More radical forms of therapy involve surgery. In general, these procedures reduce the size of the stomach and/or reroute the intestinal system to avoid the stomach. Representative procedures are gastric bypass surgery and gastric banding. These procedures can be very effective in treating obesity, but they are highly invasive, require significant lifestyle changes, and can have severe complications.

Experimental forms of treatment for obesity involve electrical stimulation of the stomach (gastric pacing) and the vagus nerve (parasympathetic system). These therapies use a pulse generator to stimulate electrically the stomach or vagus nerve via implanted electrodes. The intent of these therapies is to reduce food intake through the promotion of satiety and or reduction of appetite, and neither of these therapies is believed to affect energy expenditure. U.S. Pat. No. 5,423,872 to Cigaina describes a putative method for treating eating disorders by electrically pacing the stomach. U.S. Pat. No. 5,263,480 to Wemicke discloses a putative method for treating obesity by electrically activating the vagus nerve. Neither of these therapies increases energy expenditure.

Applicants have learned, at the cost of great time and expense, that straightforward, common sense stimulation methods often do not achieve the desired long term results. Applicants believe that the human body has several redundant systems. These systems include the redundant right and left sympathetic chains, as well as the parasympathetic nervous system including the vagus nerve, which often acts to oppose the actions of the sympathetic nerves to achieve a balance in the body. In addition, the body uses hormonal and peptide signaling through the blood stream, including signaling to the satiety and hunger centers of the brain. Some simple long term artificial stimulation may be effective at first, in the short term. In the longer term, in some systems applicants have experimentally studied, there is an initial desired response, followed by a rebound, in which the artificial stimulation is accommodated for and effectively later ignored.

What would be advantageous is a device which can be easily implanted, creates an artificial satiety signal, and which avoids being overcome by the body's accommodation response.

### SUMMARY

Stimulating the human body to reduce food consumption is not as straightforward as first believed. The human body has evolved so as to not allow a single channel of communication to over ride all other channels. Unusually non-natural signals are often detected by the body and ignored over time. In one example, a constant signal indicative of a feeling of stomach fullness may be effective at first, but if lasting a very long time, may eventually be ignored, especially if other competing signals indicate a lack of food or even hunger.

While not wishing to be bound by theory, Applicants believe several mechanisms are possible. Down regulation of some receptors of the artificially generated signals may decrease the sensitivity to such signals. Competing neural pathways may provide a more accurate indication of the state of fullness. Gut peptides and hormones indicative of hunger may be generated in the gut and reach the brain through the blood stream.

To achieve weight loss over time applicants believe that generating false satiety signals more closely tied to normal body function may escape some of the effects of habituation to the signals. Applicants believe that the duodenum is distended when food is present in the duodenum, and that this fullness may be feed back to the body to indicate satiety, and to stop eating. Applicants also believe that this sense of fullness may feed back to the stomach, perhaps through a local neural loop. This feedback to the stomach my cause the stomach to decrease peristalsis, leaving the stomach truly full of food, as the duodenum is or seems to be full of food as well. This true fullness leads to actual distension of the stomach, also causing satiety signals to be sent to the brain.

By generating false satiety signals near in time to eating a meal, Applicants believe that there may be some safe-harbor provided by effectively hiding behind a natural event, eating. In one such embodiment method, the duodenum is electrically stimulated so as to cause the nerves from the duodenal mechano receptors or stretch receptors to send signals to the brain and/or to other gut regions. In another aspect, the duodenum is stimulated electrically to cause nerves from chemo receptors to send signals to the brain or other gut regions. In various embodiments of the invention, the duodenum is stimulated so as to send fullness or food signals somewhat mimicking the naturally occurring signals. This mimicry may however occur soon after ingestion, last longer after ingestion, be of greater magnitude, and/or increase at a faster rate of change than is normally occurring. The feeling of fullness may set in earlier, last longer, and seem much larger than normal. By generating the false signals around a meal or meal times, some habituation to the false signals may be avoided.

In some embodiments of the invention, the duodenal stimulator and eating sensor are surgically placed in proximity to each other, e.g. stomach and duodenum or duodenum and duodenum. This can make surgical implantation quicker and less expensive.

### DESCRIPTION OF DRAWINGS

FIG. 1 is a diagrammatic view of an autonomic nervous system of a human, showing how the stomach and duodenum communicate through the autonomic nervous system.

FIG. 2 is a diagrammatic view of a sympathetic nervous system anatomy.

FIG. 3 is an elevation view of the splanchnic nerves and celiac ganglia.

FIG. 4 is a schematic view of an exemplary stimulation pattern having pulse trains.

FIG. 5 is a schematic diagram of an exemplary ramp-cycling or duodenal fullness mimicry treatment algorithm.

FIG. 6 shows a portion of the ramp-cycling treatment or duodenal fullness mimicry algorithm of FIG. 5 and/or the start of a stimulation dose in more detail.

FIG. 7 shows a more detailed view of a portion of the exemplary stimulation pattern of FIG. 6, showing individual pulses and a pulse train.

FIG. 8 is a schematic diagram of a human body having an implantable pulse generator implanted within and having an electrode near the duodenum.

FIG. 9 is a state diagram of logic executed in the IPG of one embodiment of the invention.

FIG. 10 is a cutaway view of a duodenum, showing the various parts.

FIG. 11 is a perspective, partially cutaway view of a duodenum having a sensor disposed near the pylorus and a helical stimulating electrode near the 3rd part of the duodenum, both coupled to an IPG.

FIG. 12 is a highly conceptual representation of the frequency response that may occur over time as the duodenum is filled and emptied of food from a normal meal.

FIG. 13 is a highly conceptual representation of the frequency response that may occur over time as the duodenum is stimulated according to some embodiments of the present invention.

FIGS. 14 through 17 are anatomical drawings having areas of particular interest highlighted using heavy lead lines, where many of the highlighted areas are arteries associated with nerves that are believed suitable as locations for electrode placement for stimulation purposes.

### DETAILED DESCRIPTION

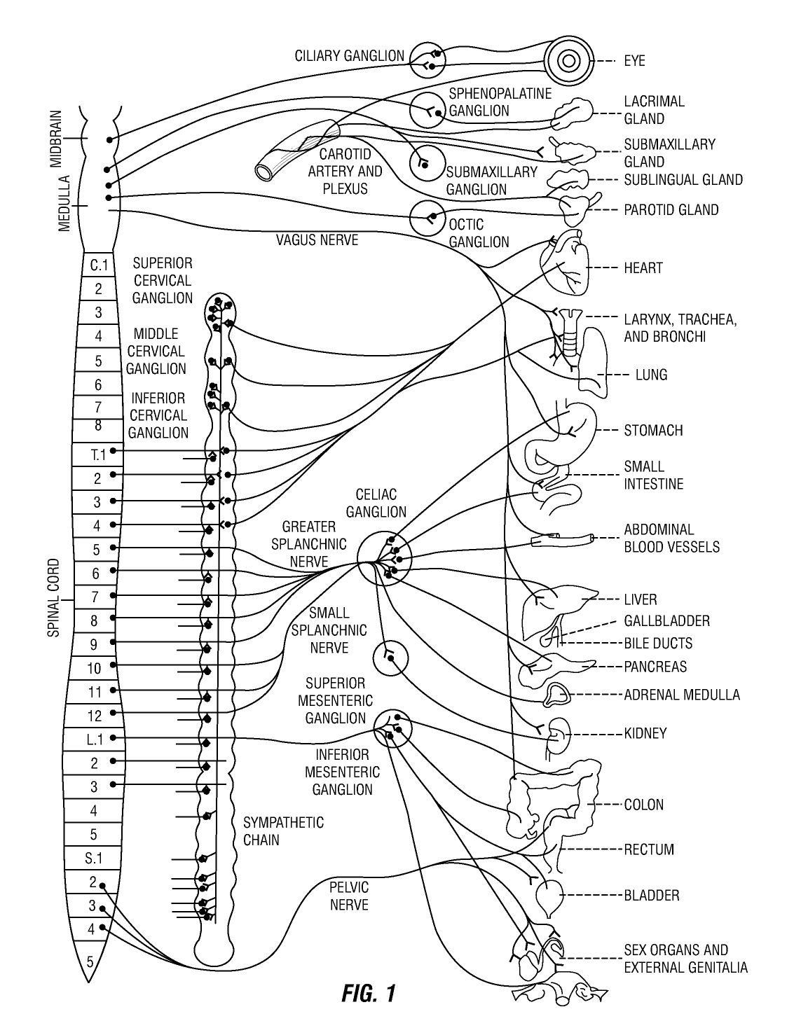

FIG. 1 illustrates the autonomic nervous system that controls involuntary actions of the smooth muscles (blood vessels and digestive system), the heart, and glands. The autonomic nervous system is divided into the sympathetic and parasympathetic systems. The sympathetic nervous system generally prepares the body for action by increasing heart rate, increasing blood pressure, and increasing metabolism. The parasympathetic system prepares the body for rest by lowering heart rate, lowering blood pressure, and stimulating digestion.

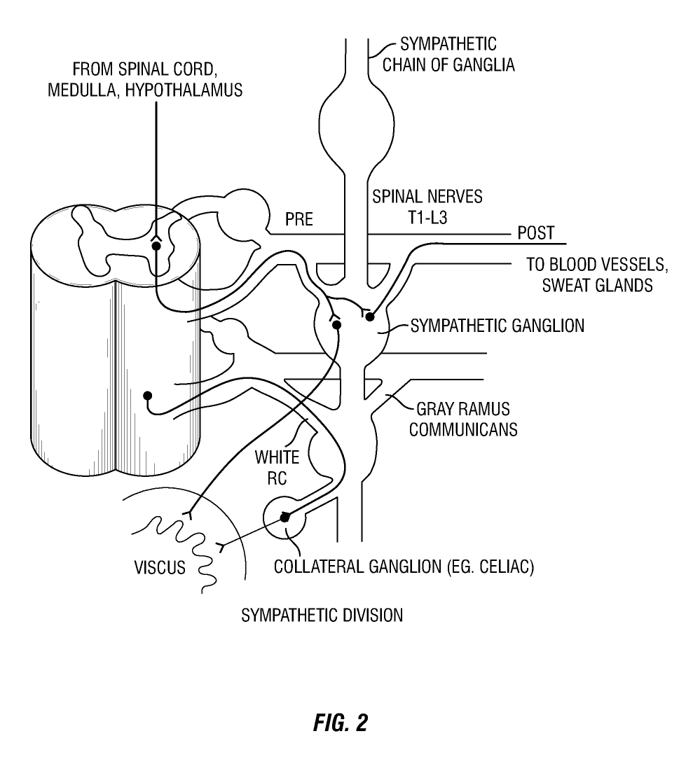

FIG. 2 illustrates the hypothalamus controlling the sympathetic nervous system via descending neurons in the ventral horn of the spinal cord. These neurons synapse with preganglionic sympathetic neurons that exit the spinal cord and form the white communicating ramus. The preganglionic neuron will either synapse in the paraspinous ganglia chain or pass through these ganglia and synapse in a peripheral, or collateral, ganglion such as the celiac or mesenteric. After synapsing in a particular ganglion, a postsynaptic neuron continues on to innervate the organs of the body (heart, intestines, liver, pancreas, etc.) or to innervate the adipose tissue and glands of the periphery and skin. Preganglionic neurons of the sympathetic system can be both small-diameter unmyelinated fibers (type C-like) and small-diameter myelinated fibers (type B-like). Postganglionic neurons are typically unmyelinated type C neurons.

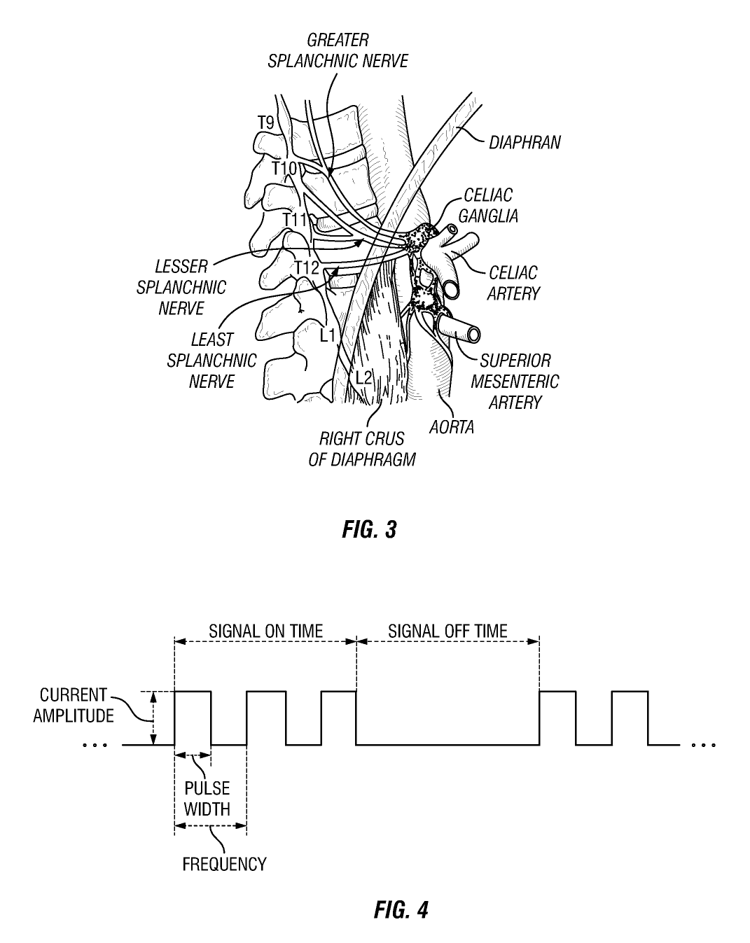

FIG. 3 illustrates several large sympathetic nerves and ganglia formed by the neurons of the sympathetic nervous system. The greater splanchnic nerve (GSN) is formed by efferent sympathetic neurons exiting the spinal cord from thoracic vertebral segment numbers 4 or 5 (T4 or T5) through thoracic vertebral segment numbers 9 or 10 or 11 (T9, T10, or T11). The lesser splanchnic (lesser SN) nerve is formed by preganglionic fibers sympathetic efferent fibers from T10 to T12 and the least splanchnic nerve (least SN) is formed by fibers from T12. The GSN is typically present bilaterally in animals, including humans, with the other splanchnic nerves having a more variable pattern, present unilaterally or bilaterally and sometimes being absent. The splanchnic nerves run along the anterior lateral aspect of the vertebral bodies and pass out of the thorax and enter the abdomen through the crus of the diaphragm. The nerves run in proximity to the azygous veins. Once in the abdomen, neurons of the GSN synapse with postganglionic neurons primarily in celiac ganglia. Some neurons of the GSN pass through the celiac ganglia and synapse on in the adrenal medulla. Neurons of the lesser SN and least SN synapse with post-ganglionic neurons in the mesenteric ganglia.

Postganglionic neurons, arising from the celiac ganglia that synapse with the GSN, innervate primarily the upper digestive system, including the stomach, pylorus, duodenum, pancreas, and liver. In addition, blood vessels and adipose tissue of the abdomen are innervated by neurons arising from the celiac ganglia/greater splanchnic nerve. Postganglionic neurons of the mesenteric ganglia, supplied by preganglionic neurons of the lesser and least splanchnic nerve, innervate primarily the lower intestine, colon, rectum, kidneys, bladder, and sexual organs, and the blood vessels that supply these organs and tissues.

In the treatment of obesity, some embodiments of treatment involve electrical activation of the greater splanchnic nerve of the sympathetic nervous system. Unilateral activation may be utilized, although bilateral activation may also be utilized. The celiac ganglia can also be activated, as well as the sympathetic chain or ventral spinal roots.

Electrical nerve modulation (nerve activation, stimulation, and/or inhibition) is accomplished by applying an energy signal (pulse) at a certain frequency to the neurons of a nerve (nerve stimulation). The energy pulse causes depolarization of neurons within the nerve above the activation threshold resulting in an action potential. The energy applied is a function of the current (or voltage) amplitude and pulse width or duration. Activation or inhibition can be a function of the frequency of the energy signal, with low frequencies on the order of 1 to 50 Hz resulting in activation of a nerve for some embodiments and high frequencies greater than 100 Hz resulting in inhibition of a nerve for some embodiments. Inhibition can also be accomplished by continuous energy delivery resulting in sustained depolarization. Different neuronal types may respond to different energy signal frequencies and energies with activation or inhibition.

Each neuronal type (i.e., type A, B, or C neurons) has a characteristic pulse amplitude-duration profile (energy pulse signal or stimulation intensity) that leads to activation. The stimulation intensity can be described as the product of the current amplitude and the pulse width. Myelinated neurons (types A and B) can be stimulated with relatively low current amplitudes, on the order of 0.1 to 5.0 mA, and short pulse widths, on the order of about 50 psec to about 200 psec. Unmyelinated type C fibers typically require longer pulse widths on the order of about 300 psec to about 1,000 psec and higher current amplitudes for stimulation. Thus, in certain embodiments, the stimulation intensity for efferent activation of a nerve may be in the range of about 0.005 mA-msec to about 5.0 mA-msec. In certain embodiments, the stimulation intensity for efferent activation of a nerve may be in the range of about 0.001 mA-msec to about 10.0 mA-msec.

The greater splanchnic nerve also contains type A fibers. These fibers can be afferent and sense the position or state (contracted versus relaxed) of the stomach or duodenum. Stimulation of A fibers may produce a sensation of satiety by transmitting signals to the hypothalamus. They can also participate in a reflex arc that affects the state of the stomach. Activation of both A and B fibers can be accomplished because stimulation parameters that activate efferent B fibers will also activate afferent A fibers. Activation of type C fibers may cause both afferent an efferent effects, and may cause changes in appetite and satiety via central or peripheral nervous system mechanisms.

Various stimulation patterns, ranging from continuous to intermittent, may be utilized for various embodiments. In certain embodiments, information related to a stimulation pattern may be stored in a storage module. For example, stimulation pattern data may be stored in volatile memory, such as random access memory (“RAM”), or in non-volatile memory, such as a hard disk drive or flash drive.

FIG. 4 illustrates an energy signal is delivered to a nerve or nerve tissue for a period of time at a certain frequency during the signal on-time. The signal on-time may be followed by a period of time with no energy delivery, referred to as a signal-off time. In certain embodiments, the signal on-time comprises a suprathreshold period, during which the energy delivered to a nerve or nerve fiber group (containing one or more nerve fibers) meets or exceeds a threshold for exciting (i.e., eliciting an action potential from) that nerve or nerve fiber group. In certain embodiments, the signal on-time comprises a subthreshold period, during which the energy delivered to the nerve or nerve fiber is below a threshold for exciting (i.e., eliciting an action potential from) that nerve (or nerve fiber group). Such a subthreshold period may comprise a period of no (or about zero) energy delivery, or an amount of energy greater than zero but less than that needed for exciting the nerve (or fiber). On average, the energy or power delivered to a nerve during a subthreshold period is greater than zero, even if there are one or more brief periods of zero-energy delivery. In certain embodiments as described herein using a signal-on time and signal-off time, a signal-on time may consist of a continuous or nearly continuous suprathreshold period. Consequently, as described herein, the effects of certain embodiments that use a signal-on time and signal-off time may be accomplished using properly configured subthreshold and suprathreshold periods during a continuous or nearly continuous signal-on time.

The ratio of the signal on-time to the sum of the signal on-time plus the signal off time is referred to as the duty cycle and it can, in some embodiments, range from about 1% to about 100%. The ratio of the suprathreshold period to the sum of the suprathreshold period plus the subthreshold period may also be referred to as a duty cycle and it can, in some embodiments, range from about 1% to about 100%. “Duty cycle” in the first definition above may be clarified as the ratio of the suprathreshold period to the sum of the suprathreshold period plus the subthreshold period (i.e., the total on-time) plus the off-time (i.e., the ratio of the suprathreshold period to the sum of the on-time and off-time). Such a duty cycle can, in some embodiments, also range from about 1% to about 100%. Peripheral nerve stimulation is commonly conducted at nearly a continuous, or 100%, duty cycle. However, an optimal duty cycle for splanchnic nerve stimulation to treat obesity may be less than 75% in some embodiments, less than 50% in some embodiments, or even less than 30% in certain embodiments. This may reduce problems associated with muscle twitching as well as reduce the chance for blood pressure or heart rate elevations caused by the stimulation energy. The on-time may also be important for splanchnic nerve stimulation in the treatment of obesity. Because some of the desired effects of nerve stimulation may involve the release of hormones, on-times sufficiently long enough to allow plasma levels to rise are important. Also, gastrointestinal effects on motility and digestive secretions take time to reach a maximal effect. Thus, an on-time of approximately 15 seconds, and sometimes greater than 30 seconds, may be used.

Superimposed on the duty cycle and signal parameters (frequency, on-time, mAmp, and pulse width) are treatment parameters. Therapy may be delivered at different intervals during the day or week, or continuously. Continuous treatment may prevent binge eating during the off therapy time. Intermittent treatment may prevent the development of tolerance to the therapy. A desirable intermittent therapy embodiment may be, for example, 18 hours on and 6 hours off, 12 hours on and 12 hours off, 3 days on and 1 day off, 3 weeks on and one week off or a another combination of daily or weekly cycling.

Alternatively, treatment may be delivered at a higher interval rate, say, about every three hours, for shorter durations, such as about 2 minutes to about 30 minutes. 30 minutes on and 60, 90 or 60-90 minutes off can be used in various embodiments. The treatment duration and frequency may be tailored to achieve a desired result. Treatment duration for some embodiments may last for as little as a few minutes to as long as several hours. Also, splanchnic nerve activation to treat obesity may be delivered at daily intervals, coinciding with meal times. Treatment duration during mealtime may, in some embodiments, last from 1 hour to about 3 hours and start just prior to the meal or as much as an hour before.

Efferent modulation of the GSN may be used to control gastric distention/contraction and peristalsis. Gastric distention or relaxation and reduced peristalsis can produce satiety or reduced appetite for the treatment of obesity. These effects may be caused by activating efferent B or C fibers at moderate to high intensities, such as about 1.0 mA to about 5.0 mA current amplitude and about 0.15 to about 1.0 millisecond pulse width and higher frequencies of about 10 Hz to about 20 Hz. Gastric distention may also be produced via a reflex arc involving the afferent A fibers. Activation of A fibers may cause a central nervous system mediated reduction in appetite or early satiety. These fibers may be activated at the lower range of stimulation intensity, for example about 0.15 msec to about 0.30 msec pulse width and about 0.1 to about 1.0 mA current amplitude and higher range of frequencies given above. Contraction of the stomach can also reduce appetite or cause satiety. Contraction can be caused by activation of C fibers in the GSN. Activation of C fibers may also playa role in centrally mediated effects. Activation of these fibers is accomplished at higher stimulation intensities, for example about 2 to about 5 times those of B and A fibers.

It should be noted that the current amplitude of a stimulation signal may also vary depending on the type of energy delivery module (such as an electrode) used. A helical electrode that has intimate contact with the nerve will have a lower amplitude than a cylindrical electrode that may reside millimeters away from the nerve. In general, the current amplitude used to cause stimulation is proportional to 1/ (Radial Distance From Nerve) 2. The pulse width can remain constant or can be increased to compensate for the greater distance. The stimulation intensity would be adjusted to activate the afferent/efferent B or C fibers depending on the electrodes used. Using the muscle twitching threshold prior to habituation can help guide therapy, given the variability of contact/distance between the nerve and electrode.

Weight loss induced by electrical activation of the splanchnic nerve may be amplified by providing dynamic nerve modulation or stimulation. Dynamic stimulation refers to changing the values of stimulation signal intensity, stimulation frequency and/or the duty cycle parameters during treatment. The stimulation intensity, stimulation frequency and/or duty cycle parameters may be changed independently, or they may be changed in concert. One parameter may be changed, leaving the others constant; or multiple parameters may be changed approximately concurrently. The stimulation intensity, stimulation frequency and/or duty cycle parameters may be changed at regular intervals, or they may be ramped up or down substantially continuously. The stimulation intensity, stimulation frequency and/or duty cycle parameters may be changed to preset values, or they may be changed to randomly generated values. In some embodiments, the changes in the stimulation signal parameters are altered through an automated process, for example, a programmable pulse generator. When random changes in the stimulation signal parameter or parameters are desired, those changes may be generated randomly by a pulse generator. One advantage of dynamic stimulation is that the patient's body is unable, or at least less able, to adapt or compensate to the changing simulation than to a constant or regular pattern of stimulation.

Weight loss induced by electrical activation of the splanchnic nerve may be improved by providing intermittent therapy, or intervals of electrical stimulation followed by intervals of no stimulation. Data shows that after an interval of stimulation, weight loss can be accelerated by turning the stimulation signal off. This is directly counter to the notion that termination of therapy would result in a rebound phenomenon of increased food intake and weight gain. This data also indicates that a dynamic, or changing, stimulation intensity (e.g., increasing or decreasing daily) produces a more pronounced weight loss than stimulation at a constant intensity. This intermittent therapy, coupled with a dynamic or changing stimulation intensity, is called the ramp-cycling technique, and ramp cycling is one subset of the dynamic stimulation techniques described herein. Given these findings, several dosing strategy embodiments are described below.

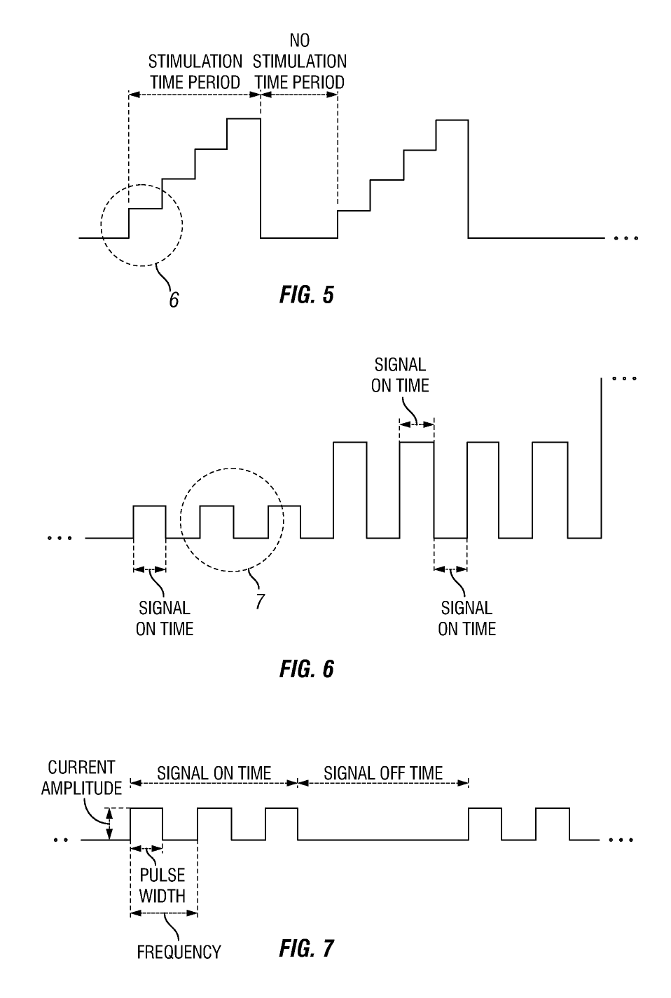

FIGS. 5-7 illustrate one embodiment of the ramp-cycling technique, shown schematically. Simulation patterns mimicking and/or augmenting duodenal filling and emptying may employ similar patterns in some embodiments. FIG. 5 has a longer time scale than FIG. 6, which in turn has a longer time scale than FIG. 7. FIG. 5 shows the main features of one embodiment of the ramp-cycling technique. Each period of the cycle includes a stimulation time period (or stimulation period) and a no-stimulation time period (or no-stimulation period). The stimulation time period may be referred to as a first time period, an interval of electrical stimulation, an interval of stimulation, a stimulation intensity ramping phase, or a stimulation interval. In certain embodiments, the stimulation time period may include on-times, offtimes, suprathreshold periods, and subthreshold periods. The no-stimulation time period may be referred to as a second time period, an interval in which the device is off or delivering low power, an interval of no stimulation, or a declining stimulation intensity period. In certain embodiments, the no-stimulation time period may include one or more subthreshold periods. The stimulation time period and no-stimulation time period should not be confused with the stimulation on-time, signal on-time (or on-period or on-time), or the signal off-time (or off-period or off-time) which are terms describing the parameters of the duty cycle and shown in FIGS. 6 and 7. The stimulation time period further comprises portions or consecutive intervals.

A single cycle of ramp-cycling therapy includes a stimulation time period and a no-stimulation time period. In some embodiments of the ramp-cycling technique, a single cycle may be repeated without changing any of the treatment parameters, the duty cycle parameters or the signal parameters of the original cycle. In certain embodiments the treatment parameters, and/or the duty cycle parameters and/or the signal parameters may be changed from cycle to cycle. In certain embodiments, a single cycle of ramp-cycling therapy comprises one to many suprathreshold periods and subthreshold periods.

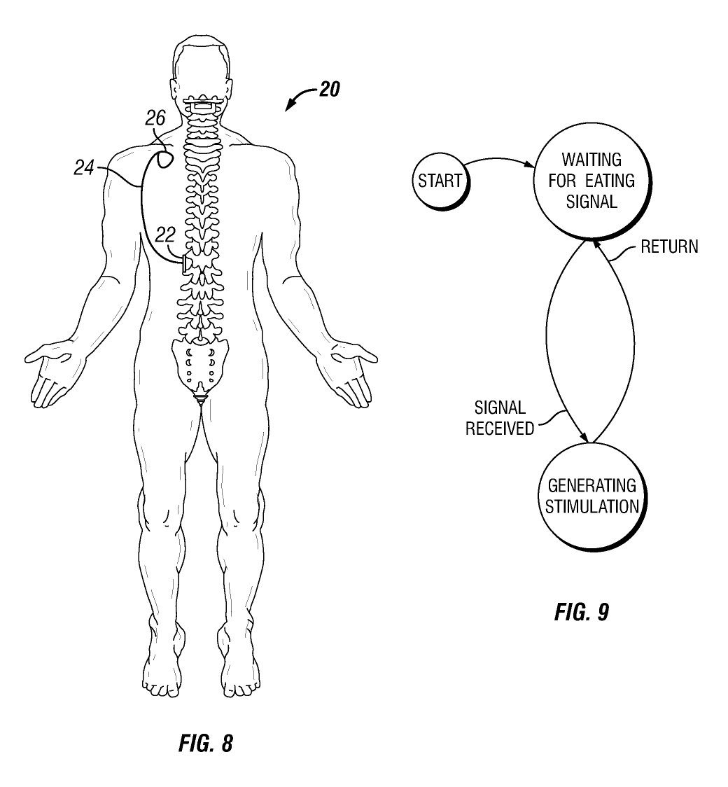

FIG. 8 illustrates a schematic view of an IPG implanted within a human body. The IPG can be a neurostimulator which may be similar in some respects to existing neurostimulators. In this illustration, the IPG has an output coupled to a nerve cuff which is positioned over the duodenum. Various electrodes may be used in various embodiments, including but not limited to cuff electrodes, patch electrodes, monopolar, bipolar, tripolar, and quadrapolar electrodes. In some embodiments, the housing of the IPG can serve as one of the electrodes. For examples in which the lead is placed within a vein a monopolar lead is usually used. A sensor measuring a property indicative of eating may also be coupled to the IPG in some embodiments.

In some embodiments, the current supplied can vary in current intensity from about 0 mA to about 10 mA, in increments. Some IPGs output pulse trains having a number of pulses having a frequency which can vary from about 1 Hz to about 40 Hz. Some devices allow for the ramping of current and/or frequency.

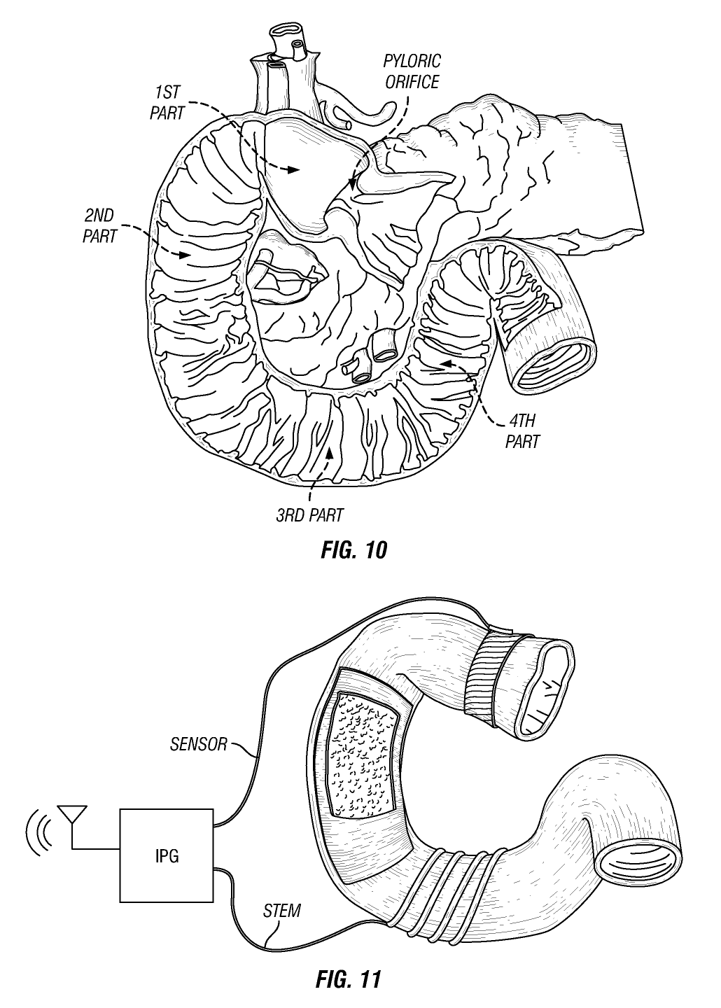

FIG. 9 illustrates one example of logic which can be executed in one embodiment of the invention. In a WAITING FOR SIGNAL state the IPG can wait to receive a signal indicative of eating. This signal can come from various sources in various embodiments. In one embodiment, the signal may be generated by the patients themselves using a magnet or patient programmer unit. In this embodiment; the signal that eating is to begin can be manually input in some embodiments. This can be used to immediately trigger duodenal stimulation or to do so after a time delay. In various embodiments, the signal may be generated by the esophagus, the stomach, the duodenum, autonomic nerves, and combinations thereof. In one embodiment, the splanchnic or vagal nerve may be monitored for signs of eating. This signal may be filtered and otherwise cleaned up, to detect whether eating activity is taking place.

The GENERATING STIMULATION state can then be entered. In this state a pattern may be generated, in some embodiments, which mimics a natural waveform as might be delivered from the duodenum. In one such example, the duodenum might be expected to send an increasing frequency signal over 20-60 minutes, followed by a plateau for 10 minutes, followed by a decrease infrequency and also possibly current intensity. In the body, the duodenum may generate these signals to the brain directly, as pressure and chemoreceptor nerve outputs. This signal may also travel a shorter path, from the duodenum to the stomach in a small, local neural loop, to urge the stomach to slow peristalsis until it is sensed that food has cleared the duodenum. When using an artificial pulse generator, a wave form pattern may begin sooner after the signal is received than normal, providing a false sense of duodenal fullness earlier than normal. In some embodiments, a falsely high rate of increase of frequency may provide a false sense of duodenal rapid filling. The sense of fullness may also be extended longer than normal, falsely indicating a large meal. Electrically stimulating the duodenum may thus stimulate mechano receptors or nerves from these receptors indicating duodenal distension, and may also stimulate nerves coupled to chemoreceptors indicative of the presence of certain nutrients.

The stimulation waveform to the duodenum can be a constant stimulation intensity and frequency in some embodiments, while varying one or both as described above in other embodiments. In this way, the duodenal distension and emptying cycle signals may somewhat resemble the ramp cycling previously discussed.

After the desired stimulation is over, the WAITING FOR SIGNAL STATE can be re-entered.

In some examples a handheld patient programmer device can be used. This device can communicate with the IPG using telemetry through inductive coupling.

The device can have three buttons which may be pressed by the patient. The lower button can be the STATUS button, which may be used to query the IPG to transmit the device status, which is indicated by the 4 upper status lights and also the upper left CALL PHYSICIAN light. The middle button is the DOSE button, which instructs the IPG to deliver a dose of therapy. In some embodiments, this dose is a modified or exaggerated meal response pattern. As previously discussed, this dose can be applied to the duodenum to generate neurological signals from the duodenal nerves to provide artificial duodenal fullness signals. This dose, in one embodiment, is a dose having a profile, length, frequency, and maximum current set in the IPG by a medical professional. As long as the dose is being delivered, the DOSE light will be the status returned by the IPG. The SUSPEND button may be pressed, in one embodiment, to serve the same function as the magnet placement. The SUSPEND light will show a suspend status for a certain time period e.g. 30 minutes after the IPG was instructed to suspend, either by the magnet or the patient programmer.

FIG. 10 illustrates a cross-sectional cutaway view of a duodenum, showing the four parts and the pylorus.

FIG. 11 shows one example of one embodiment of the present invention, having a sensor secured near the pylorus for sensing activity indicative of eating, a stimulating electrode secured near the third part of the duodenum, with both being coupled to an IPG. The sensor can be placed many other locations, including but not limited to the esophagus, the stomach, the duodenum, the splanchnic nerve, etc.

In some embodiments, an eating sensor and the duodenal stimulator are surgically placed relatively near to each other. In one example, the sensor would be placed near the esophagus or stomach and the stimulator electrode near the duodenum. In another example, the sensor would be placed on the pylorus and/or first part of the duodenum, and the stimulator electrode placed on the first, second, third, and/or fourth part of the duodenum.

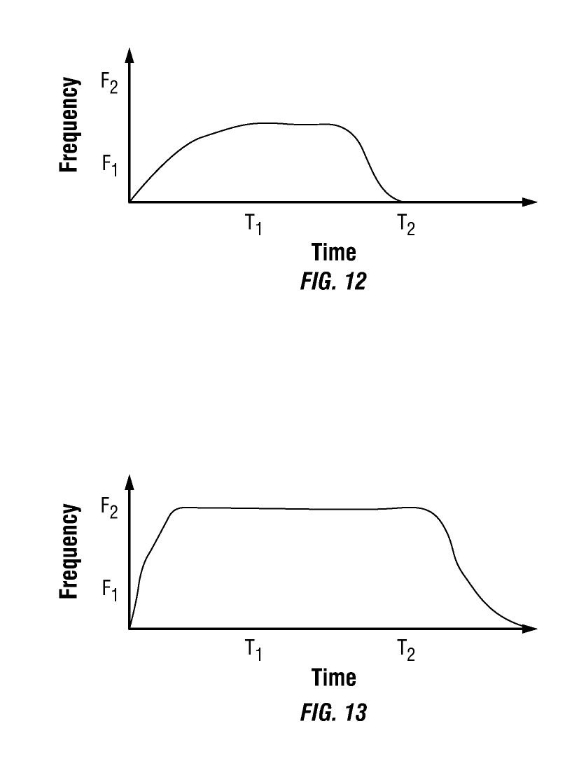

FIG. 12 is a highly conceptual representation of the frequency response that may occur over time as the duodenum is filled and emptied of food from a normal meal. As the food fills the duodenum, the frequency increases over time, reaching a peak frequency of f1 and time t1, and then decreasing back to baseline frequency at time t2.

FIG. 13 is a highly conceptual representation of the frequency response that may occur over time as the duodenum is stimulated according to some embodiments of the present invention. In this example, the frequency at the duodenal autonomic nerves rises more quickly and to a higher frequency than the natural response in FIG. 12. The peak frequency f2 is hit much faster than was the peak frequency in FIG. 12. In addition, the peak frequency f1 is less than f2. The frequency plateau lasts longer in FIG. 12 than in 13. The frequency response of FIG. 13 takes much longer to return to baseline in the artificially generated result of FIG. 13 than in the natural case of FIG. 12.

The present invention can thus provide various methods for inducing satiety. One method includes electrically stimulating the autonomic and/or enteric nervous system near the duodenum by applying an electrical stimulation pattern which induces a biological signal indicating duodenal distension and/or the presence of food in the duodenum, where the electrical stimulation pattern includes a plurality of electrical signals over time. In some methods, the biological signal includes biological signals that communicate primarily afferently rather than efferently. The electrical signals may recruit a substantially larger portion of A fibers than B fibers. The electrical signals have a frequency of between about 1 Hz and 40 Hz, 1 Hz and 30 Hz, 1 Hz and 20 Hz, and 1 Hz and 10 Hz, in various embodiments of the invention.

In some embodiments, the electrical signals have a frequency which increases over a period of time mimicking a period of normal stomach filing during a normal meal. In other embodiments, the frequency increases over a period of time faster that of normal stomach filing during a normal meal. In still other embodiments, the frequency increases over a period of time at least twice as fast as that of normal stomach filing during a normal meal.

In some embodiments, the stimulating includes delivery using an electrode wrapped around at least part of the nerves on the duodenum. The stimulating may include delivery using an electrode wrapped around the first part of the duodenum, in some methods. In various other methods, the stimulating includes delivery using an electrode wrapped around the second, third, or fourth parts of the duodenum, and combinations thereof.

In some methods, the signal is delivered at times corresponding to typical meal times. In other methods, the signals are delivered responsive to signals indicative of eating. The signals are indicative of stomach distension in still other embodiment methods. The signal includes a manually generated signal in some embodiments. The signal includes an esophageal muscle signal and/or a local reflex duodenal relaxation signal in various other embodiments. The signal includes a duodenal signal received directly from a pyloric sphincter muscle indicative of relaxation in some methods. The signal can include a local reflex nerve duodenal contraction signal.

In some methods, the frequency increases in frequency at least about 10 Hz over the course of between about 1 minute and one hour, responsive to an expectant normal meal time and/or indication of feeding. The method may be followed by a substantial decrease in stimulation within at least 2 hours of the onset of stimulation.

Biological signals generated by the present electrical stimulation methods can include nerve signals, gut hormones, and/or gut peptides.

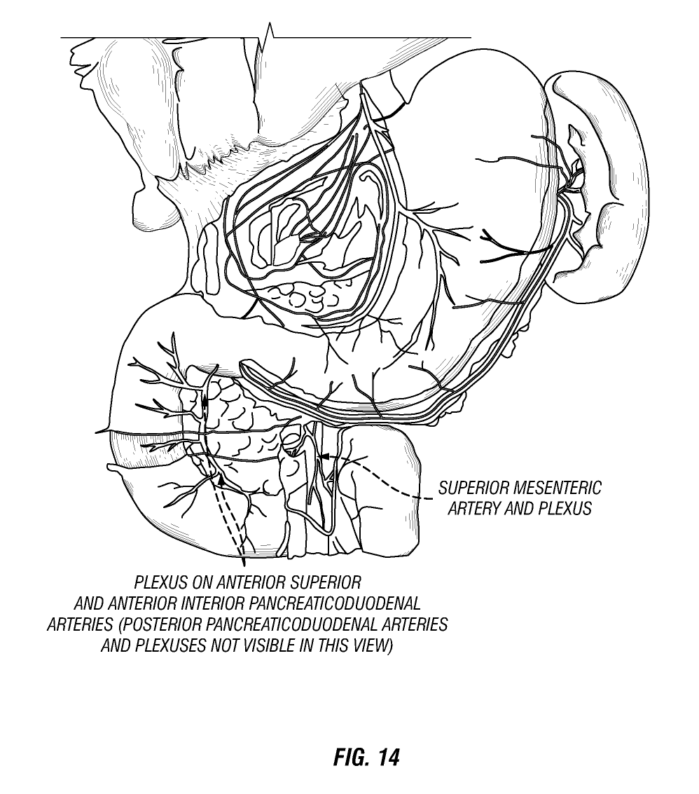

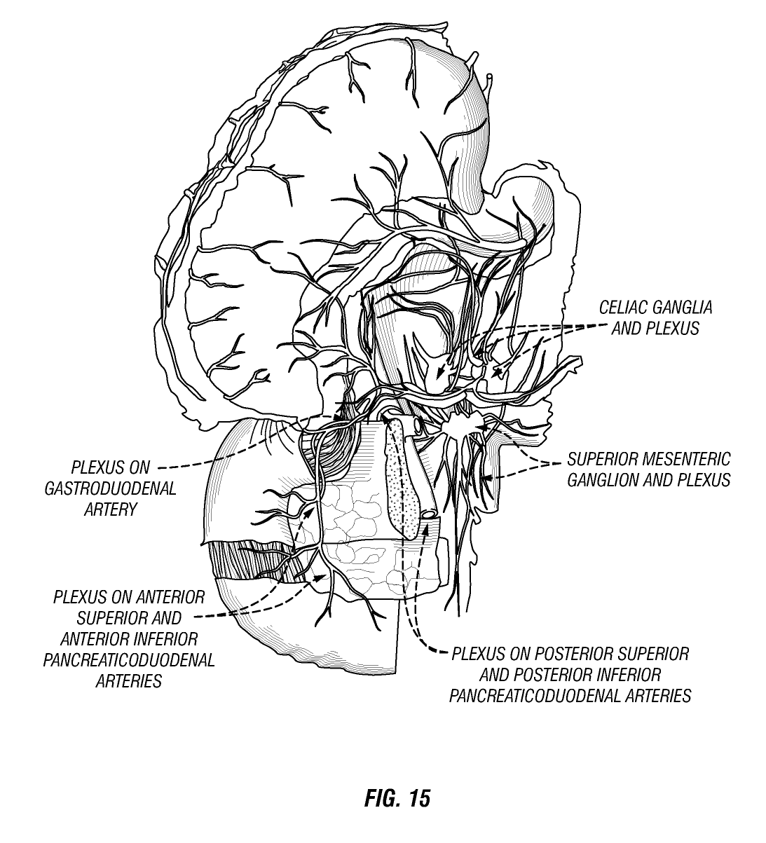

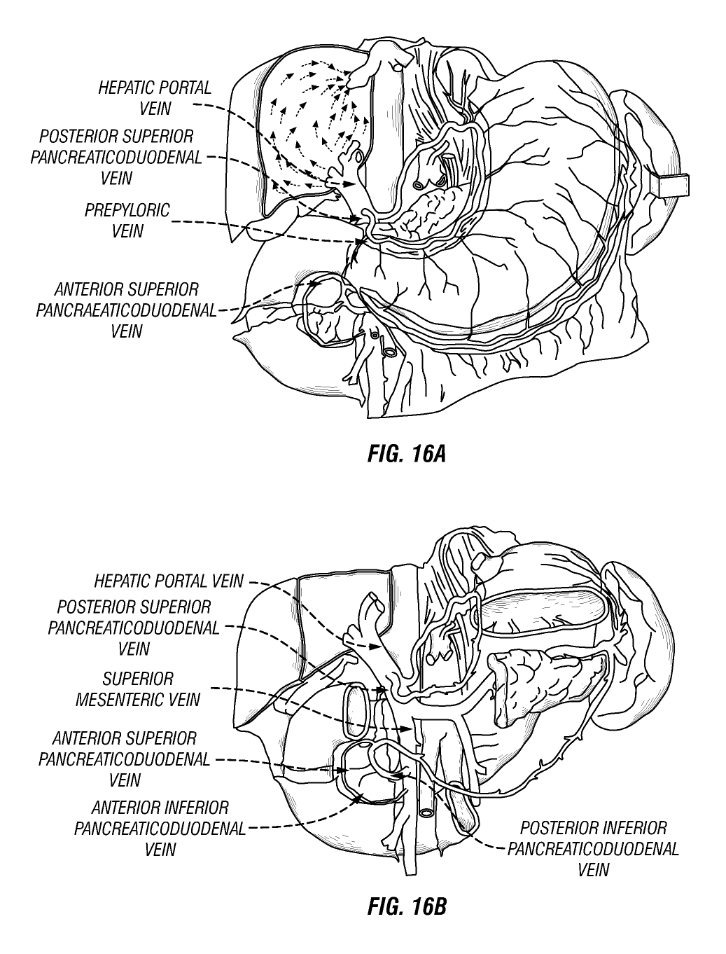

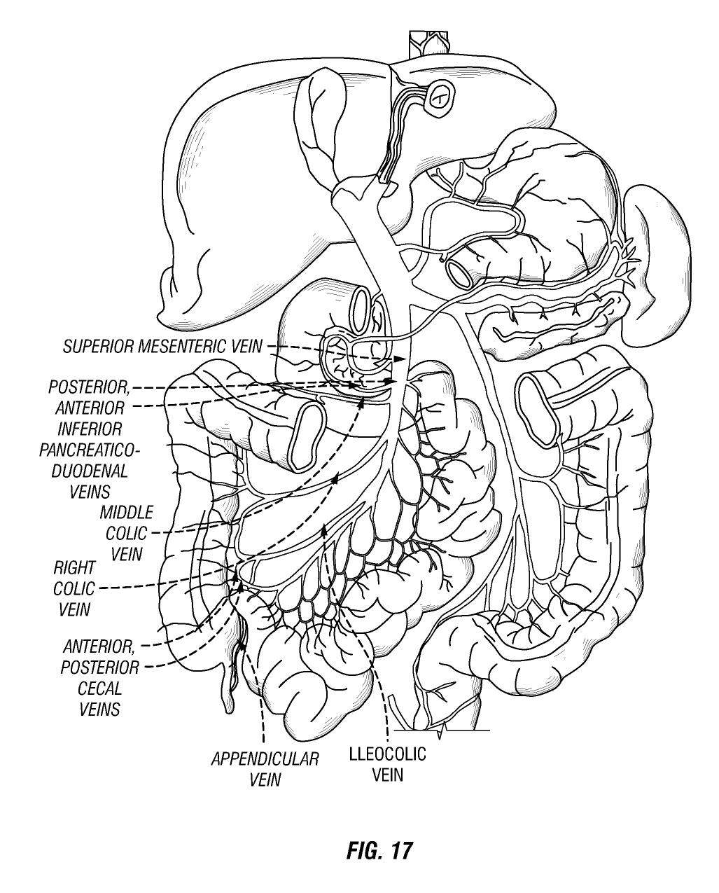

FIGS. 14-17 show the anatomy near the duodenum, particularly the innervations of the duodenum. The nerves innervating the duodenum typically follow the arteries that supply blood to the area. In some embodiments, the duodenum is stimulated and optionally sensed using nerves which innervate the duodenum. As these nerves often travel with blood vessels, electrodes may be disposed near, in, and/or around blood vessels carrying such nerves. In one embodiment, stimulating includes stimulating autonomic nerve fibers caudal to the mesenteric plexus, enteric plexus, hepatic plexus, right gastric plexus, nerves of anterior, superior and inferior pancreaticoduodenal. In various embodiments, stimulating includes stimulating at nerves which are disposed along at least one of the superior mesenteric vein, posterior, anterior, inferior pancreaticoduodenal veins, middle colic vein, right colic vein, ileocolic vein, anterior, posterior cecal veins, hepatic portal vein, posterior superior pancreaticoduodenal vein, prepyloric vein, anterior superior pancreaticoduodenal vein, hepatic portal vein, posterior superior pancreaticoduodenal vein, superior mesenteric vein, anterior superior pancreaticoduodenal vein, anterior inferior pancreaticoduodenal vein, posterior inferior pancreaticoduodenal vein and/or combinations thereof. Electrodes can be placed transvascularly within one or more of the veins that are near or on the duodenum.

What is claimed is:

1. A method for inducing satiety, the method comprising:
electrically stimulating the autonomic and/or enteric nervous system of a duodenum by applying an electrical stimulation pattern which induces a biological signal indicating duodenal distension and/or the presence of food in the duodenum, where the electrical stimulation pattern includes a plurality of electrical signals over time.

2. The method of claim 1, in which the biological signal includes biological signals that communicate primarily afferently rather than efferently.

3. The method of claim 1, in which the electrical signals recruit a substantially larger portion of A fibers than B fibers.

4. The method of claim 1, in which the electrical signals have a frequency of between about 1 Hz and 20 Hz.

5. The method as in claim 4, in which the electrical signal frequency increases over a period of time mimicking a period of normal stomach filing during a normal meal.

6. The method as in claim 5, in which the frequency increases over a period of time at least twice as fast as that of normal stomach filing during a normal meal.

7. The method of claim 1, in which the signal is delivered at times corresponding to typical meal times.

8. The method of claim 1, in which the signals are delivered responsive to signals indicative of eating.

9. The method of claim 1, in which the electrical stimulation is a manually generated signal.

10. The method of claim 1, wherein the electrical stimulation is provided by at least one electrode that is positioned within a vein near the duodenum.

11. The method of claim 10, in which the signal includes a signal from a pressure sensing sleeve disposed around the duodenum.

12. The method of claim 1, in which the biological signal generated includes a nerve signal.

13. The method of claim 1, in which the biological signal generated includes a gut hormone or peptide signal.
